(19) United States
(12) Patent Application Publication
(10) Pub. No.: US 2004/0180087 A1
(43) Pub. Date: Sep. 16, 2004

(54) Stable controlled release pharmaceutical compositions containing pravastatin

(76) Inventors: Boyong Li, Morgantown, WV (US); Aaron Dely, Fort Lauderdale, FL (US); David Wong, Milpitas, CA (US)

Correspondence Address: Martin P Endres, Hedman & Costigan, 1185 Avenue of the Americas, New York, NY, 10036-2601, US

(21) Appl. No.: 10/479,817

(22) PCT Filed: Jun. 5, 2002
(86) PCT No.: PCT/US02/17589

Related U.S. Application Data
(60) Provisional application No. 60/300,027, filed on Jun. 21, 2001.

(51) Int. Cl. A61K 9/24

(52) U.S. Cl. 424/471; 424/473

(57) ABSTRACT

Stabilized sustained release pharmaceutical preparations containing a drug which is sensitive to a low pH environment, such as pravastatin are disclosed in which pravastatin degradation is prevented with a buffering agent. The basic excipient enhances storage stability.

### BACKGROUND OF THE INVENTION

1. Field of the Invention

A pharmaceutical composition is provided for a medicament which is sensitive to a low pH environment of less than 3, such as pravastatin. Novel, stable oral dosage formulations of pravastatin are provided which include a buffering agent to stabilize and maintain the pH below 9. As used herein the term pravastatin refers to the free base form of the drug as well as pharmaceutically acceptable salts.

2. Description of the Related Art

Pravastatin sodium, designated chemically as [1S-[1α(βS*,δS*), 2α,6α, 8β-(R*),8aα]]-1,2,6,7,8,8a-hexahydro-β,δ,6-trihydroxy-2-methyl-8-(2-methyl-1-oxobutoxy)-1-naphthaleneheptanoic acid monosodium salt is a hydroxymethylglutaryl-CoA (HMG-CoA) reductase inhibitor antilipemic agent described in U.S. Pat. No. 4,346,227. It is indicated in hypercholesterolemic patients for primary prevention of coronary events including myocardial infarction (MI); to reduce the risk of undergoing myocardial revascularization procedures; to reduce the risk of cardiovascular mortality. It is indicated in hypercholesterolemic patients for secondary prevention of cardiovascular events, including MI and to slow the progression of coronary atherosclerosis. Pravastatin is also used as an adjunct to diet for the reduction of elevated total- and LDL-cholesterol and triglyceride levels in patients with primary hypercholesterolemia and mixed dyslipidemia (Types IIa and IIb). The agent specifically competitively inhibits 3-hydroxy-3-methyl-glutaryl-coenzyme A (HMG-CoA) reductase, the enzyme that catalyzes the conversion of HMG-CoA to mevalonate, which is an early rate-limiting step in cholesterol biosynthesis. HMG-CoA reductase inhibitors increase HDL cholesterol and decrease LDL cholesterol, VLDL and plasma triglycerides. The usual dosing regimen is 10-40 mg once daily at bedtime.

Some drugs are not stable by themselves or in an acid environments and certain drugs require an alkaline environment for the purposes of stability. Stability requirements are covered in the United States Pharmacopoeia (U.S.P.), in the Good Manufacturing Practices (GMPs) as well as in FDA Guidelines for stability studies. Buffers may be added to increase stability of certain pharmaceuticals. Buffers may also increase the thermo stability of the drug in formulations that require drying during the process of producing the final dosage foam.

Pravastatin sodium is relatively polar hydrophilic, acid labile, and degrades to form its lactone and various isomers. Degradation results in lower bioavailability of pravastatin sodium. Pravastatin sodium requires a buffer to enhance storage stability. Strategies used in the prior art to stabilize pravastatin sodium formulations include: addition of a basifying agent to raise the pH to at least 9, and packaging of the product in a manner to decrease exposure to moisture.

The stability of pravastatin sodium is affected by factors including formulation and storage conditions. Pravastatin sodium is known to be an acid labile compound. Labeling of pravastatin sodium tablets indicates storage at a temperature not to exceed 30° C. and protection from light and moisture.

Stabilization is achieved by basification of the environment in which degradation occurs. Stabilized compositions in the prior art have a pH of 9 or over in an aqueous dispersion. The amounts of basifying agent range from 1 to 75%. Basifying agents used include magnesium oxide. Patents such as U.S. Pat. Nos. 5,180,589, 6,235,311, 5,225,202, 5,030,447 (which are incorporated herein by reference) describe aluminum oxide, all alkali metal hydroxides such as sodium hydroxide, potassium hydroxide or lithium hydroxide, or alkaline earth metal hydroxides such as calcium, magnesium, aluminum hydroxide, dihydroaluminum sodium carbonate, aluminum magnesium hydroxide sulfate, aluminum hydroxide magnesium carbonate co-dried gel, or ammonium hydroxides, calcium carbonate, magnesium carbonate, magnesium stearate, piperazine, sodium acetate, sodium citrate, sodium tartrate, sodium maleate, sodium succinate and mixtures thereof. Stabilization of the commercially available pravastatin sodium (Pravacol®) is achieved by basification by magnesium oxide which imparts a pH above 9, preferably about 10.

Although the prior art basifying agents can prevent the degradation of pravastatin sodium, they are less desirable because some are strong bases which may have an adverse effect on excipients used with pravastatin sodium pharmaceutical compositions. For example, lactose discolors and emits a caramelized odor in the presence of certain basifying agents, for example piperazine. Additionally, the high alkalinity occurring at dissolution of these formulations may disrupt the acidic pH milieu of the gastrointestal (GI) mucosa and is problematic for patients with pre-existing GI mucosal damage.

The need exists for a stable pravastatin formulation. The need exists for a stable controlled release formation of pravastatin. Such formulations have a practical application, and represent a valuable contribution to the medical arts. The preset invention provides such coin positions, and offers efficient and cost effective methods of preparation.

### SUMMARY OF THE INVENTION

The present invention meets the unfulfilled needs of the pharmaceutical industry.

The foregoing objectives are met by a pharmaceutical composition which has enhanced stability comprising:

(a) a core comprising:

(i) pravastatin;

(ii) an osmagent;

(iii) a buffing agent; and

(b) a semi permeable membrane with a passageway to release the drug at a desire rate, and a plasticizer incorporated into said semi permeable membrane.

In accordance with the present invention, the formulation for stable pravastatin is controlled release. Controlled-release dosage forms can provide a more constant level of the drug over a prolonged period of time. Any known technique known in the prior art for preparing sustained or controlled release pharmaceutical dosage forms may be employed. One common technique involves surrounding an osmotically active drug core with a semipermeable membrane or wall. The drug is released from the core over time by allowing gastric or intestinal fluid to permeate the coating membrane and dissolve the drug so the dissolved drug can permeate the membrane. In some cases a hydrogel is employed to push the active ingredient through a passageway in the membrane. The materials for the semi-permeable membrane are commonly known in the industry and described in U.S. Pat. Nos. 5,082,668, 4,783,337, 4,612,008 and 4,327,725 which are incorporated herein by reference.

Other common techniques in the art for preparing controlled release pharmaceutical formulations are to encapsulate or tablet a plurality of pellets, granules, or beads that are coated or to prepare matrix systems that are with sustained release coating materials such as are described in U.S. Pat. Nos. 5,286,497 and 5,002,776 which are incorporated herein by reference; or to prepare matrix systems such as described in WO 00/04879.

The pharmaceutical formulation of the present invention is a solid dosage form of a medicament having a unique buffer system which results in excellent stability. Specifically, the current invention is directed to stable controlled release oral dosage form formulations of a medicament sensitive to a low pH environment, such as pravastatin, one or more fillers such as mannitol, one or more plasticizers such as polyethylene glycol, one or more lubricants, such as magnesium stearate, one or more coloring agents, such as Opadry White, and one or more buffering agents, such as tromethamine or sodium phosphate dibasic to impart a pH to an aqueous dispersion of the composition of less than 9.0, preferably less than 8.5, and most preferably less than 8.0.

A preferred formulation can be made by addition of tromethamine or sodium phosphate dibasic in a range of 1-10%, preferably 1-5% as a buffering agent which imparts an increase in localized pH within the composition. This increase in localized pH prevents the pravastatin sodium from degrading to form its lactone and various other isomers. The pH of the aqueous dispersion of the composition is about 8 but does not exceed 9 in an aqueous environment. The composition also exhibits excellent stability when stored under accelerated conditions of 40° C. and 75% relative humidity.

Accordingly, it is an object of this invention to provide a novel and useful sustained release pravastatin formulation which is stable in a low pH environment. This represents an unexpected improvement in the art and substantially overcomes the disadvantages known to the prior art.

It is also an object of the present invention to provide both a method of stabilizing pravastatin to slow the degradation thereof and provide products that can be stored for long periods of time at room temperature, i.e. under humidity and temperature conditions usually encountered in pharmacies and medicine cabinets. It is a further object to provide solid oral pravastatin dosage forms where the amount of active drug will be prevented from being reduced to less than 90% of its labeled strength, and more preferably not less than 95% of the labeled strength after one year of storage under controlled room temperature conditions.

Other objects, features and advantages of the invention are not taught in the prior art but will be more apparent to those versed in the art from the following specification, taken in conjunction with the accompanying claims.

### DETAILED DESCRIPTION OF THE PREFERRED EMBODIMENTS

In accordance with the present invention, a pharmaceutical composition is provided which includes a medicament which may degrade in a low pH environment but which is prevented from doing so by the addition of a buffering agent. Accordingly, the pharmaceutical composition of the invention includes drugs which are chemically unstable in an acidic environment, such as pravastatin.

The invention provides sustained-release pravastatin formulations which provide alternatives to the prior art formulations which require the presence of a basifying agent which have a pH of at least 9.

Unlike the prior art basifying agent requirement, the invention favorably influences stability by the addition of a buffer which can be an alkaline reacting organic compound, a hydroxide of an alkali metal, an alkaline salt of phosphoric acid, carbonic acid or silicic acid or an alkaline ammonium salt. Representative examples of these buffers are described in U.S. Pat. No. 6,013,281 which is incorporated herein by reference. Basifying agent, as the term is used herein, refers to compounds capable of raising the pH to above 7. They are added to formulations of pravastatin to improve chemical and physical stability. According to previous pravastatin formulations containing basifying agents, tablets should retain 80-90% of active ingredient at the end of one year in the presence of stabilizers.

The stability of these formulations without a basifying agent was tested in accordance with and exceeding current pharmaceutical industry standards for storage (i.e., four to twelve weeks at about 40° C. and about 75% relative humidity). Formulations of the present invention stored under these conditions retain at least 90% of the pravastatin in the composition at the time of storage. Standard procedures such as HPLC or UV spectroscopic methods may be used to determine the amount of active ingredient remaining after storage. The final dosage form most preferably retains assay limits of 90 to 110 percent of the original assay value when stored under controlled room temperature conditions. The design of the stability studies was in compliance with the general requirements suggested by the FDA stability guidelines.

The total amount of inactive ingredients in the formulations is preferably 30% or more of the weight of the pravastatin. The tablets are prepared by the direct compression method.

The invention is particularly adaptable to pharmaceutical compositions containing pravastatin sodium. Pharmaceutical compositions of the present invention generally contain 10-40 mg or an amount with the range of about 2 to about 50% of pravastatin by weight, and preferably from about 4 to about 25% by weight of the composition. More preferred compositions of the invention contain 40 mg of active ingredient and may be in the form of tablets, caplets or capsules.

The pharmaceutical formulations of the present invention provide a stable environment for drugs which require an alkaline environment by utilizing a buffer. The formulations contain a buffering agent present in an amount within the range of about 0.5 to about 10% by weight of the composition. A preferred buffer is tromethamine. Examples of other suitable buffering agents include sodium acetate, sodium citrate, sodium tartrate, sodium fumarate, sodium maleate, sodium succinate, combinations of sodium or potassium hydroxide with sodium or potassium acid phosphate.

A preferred buffering agent is tromethamine, a weak base amino-alcohol also known as 2-amino-2 hydroxymethyl-1,3-propanediol, (tris(hydroxymethyl)aminomethane) or TRIS. Tromethamine has a greater buffering capacity than bicarbonate; pKa 7.82 versus 6.1, respectively. Tromethamine has been found to have excellent stabilizing effects on solid dosage forms containing drugs with limited water solubility which need to be solubilized in buffer to avoid otherwise solubilizing the drug in large quantities of granulating media. Tromethamine has been discovered to be most advantageous when a therapeutically-effective buffer-soluble drug has a solubility at 25° C. of less than 1 mg of drug per ml of water at pH 7.0 or lower. An advantage of tromethamine lies in its water solubility and, accordingly, it blends well with an excipient like lactose. Tromethamine as used herein is preferably present in the range of about 1 to 10%, more preferably, 2 to about 6% of the pravastatin sodium drug granulation, and most preferably 4% by weight of the condition.

Another preferred buffering agent is dibasic sodium phosphate (Na₂HPO₄), which is very soluble in water and widely used as a buffering agent for pharmaceuticals. Dibasic sodium phosphate as used herein is preferably present in the pravastatin sodium granulation in the range from about 0.5 to 10%, more preferably, 1 to about 5%, and most preferably, 2.5% by weight of the composition.

A preferred controlled release formulation is an osmotic tablet as described in Example 1 below and may contain one or more osmagents in a range from about 30 to about 95% by weight and preferably from about 50 to about 80% by weight. Mannitol which is considered an inert pharmaceutical excipient is added as a directly compressible tableting excipient. Mannitol is also used as a diluent to achieve content uniformity of the finely divided active ingredients. Mannitol functions as an osmotically effective osmagent which is soluble in the fluid that enters the tablet via a drilled hole, and exhibits a pressure gradient across the semipermeable wall against the exterior fluid. In the preferred embodiment, the amount of mannitol is 80-85% of the granules, and 65% of the total tablet weight. Examples of other suitable osmagents known to those skilled in the art include, sucrose, dextrose, cellulose derivatives such as hydroxypropyl methylcellulose, microcrystalline cellulose, povidone, calcium carbonate, calcium sulfate, magnesium carbonate, potassium sulfate, sucrose, sorbitol, sodium chloride and mixtures thereof also may be used.

An effective amount of any generally accepted pharmaceutical tableting lubricant, may be added to compress the tablets. An amount within the range from about 0.1 to about 6%, preferably 0.5 to about 2% by weight may be added. Tablet lubricants present as are preferably from the group consisting of glyceryl monostearates, magnesium stearate, palmitic acid, talc, carnauba wax, calcium stearate, sodium or magnesium lauryl sulfate, calcium soaps, zinc stearate, polyoxyethylene monostearates, calcium silicate, silicon dioxide, talc, hydrogenated vegetable oils and fats, or stearic acid. Most preferably, magnesium stearate is present as a lubricant to prevent the powder from agglomerating during processing on a high speed rotary press. Magnesium stearate is added to the granulation to assist compression. A preferred lubricant is magnesium stearate. In the preferred embodiment in Example 1, magnesium stearate is used in an amount of less than 2% of the tablet.

Controlled release occurs via a wall formed of a material that does not adversely affect the drug but which is permeable to the passage of an external aqueous fluid. The tablets of the invention include a semipermeable membrane. In one embodiment, semipermeable membrane is comprised of cellulose acetate, which may be 2-30% of the total formulation, preferably 15-20%. Other typical materials for forming the wall may include, cellulose aceylate, cellulose diacylate, cellulose triacylte, cellulose, agar acetate, polyamides, cellulose acetate phthalate, polyamides, polyurethanes.

A plasticizer is added to the semipermeable film coating. Suitable plastacizers can be added from 0.5-20% by weight of the coating composition and at least one can be selected from diethyl phthalate, citrate esters, polyethylene glycol, glycerol, acetylated glycerides, castor oil and the like. Polyethylene glycol is a preferred plasticizer and is used at a concentration of 1-5% of the coating.

Drug is released through a “passageway” means suitable for releasing the drug from the system. In the present invention, a bore or other orifice has been drilled therethrough. A detailed description of osmotic passageways and the maximum and minimum dimensions for a passageway are disclosed in U.S. Pat. Nos. 3,845,770 and 3,916,899, the disclosures of which are incorporated herein by reference. The drilled passageway connects the layer of drug plus osmagent with the exterior of the tablet.

The tablets are then seal coated. These seal coats may be comprised of low viscosity hydroxypropyl, cellulose, low viscosity hydroxypropyl methyl cellulose, carageenan, alginates, and other materials known in the art. A preferred seal coat are materials sold under the name Opadry® (Colorcon, West Point, Pa.). The coating may be 1-5% of the total tablet weight. Additionally, up to 0.5% of Candililla wax powder may be added as a polishing agent.

Method of Manufacture

Pravastatin is granulated with the osmagent and the buffer solution. The pravastatin granules preferably comprise 50-90% of the total tablet weight, more preferably 60-80%, and most preferably 70-75%. The preferred pravastatin granule composition of the invention is given below:

Pravastatin granules

| | Weight % | Weight % |
|---|---|---|
| Ingredient | granules | tablet |
| Pravastatin | 5-25 | 1-15 |
| Osmagent | 30-90 | 50-70 |
| Buffering Agent | 1-10 | 2-6 |

The manufacture of tablets of the present invention comprises the steps of:

(a) Dissolving a buffer in water, solvent or any combination,

(b) Mixing the dry materials comprising pravastatin and an osmagent; and

(c) Granulating the mixture.

The osmagent (preferably mannitol) is separately screened or milled to break up agglomerates. The screened material and the drug are combined as a dry mixture. A solution containing a dissolved buffer is used to granulate the dry mixture until desired consistency of granule is achieved. The granules are then passed through a 25 mesh screen, dried by conventional methods and passed through a Fitzmill. The drug granulation is then blended with sufficient quantity of filler to bulk up for tablet compression. The filler is screened through a 25 mesh screen. The drug granules are placed into a blender with screened filler. The lubricant is then screened and added to the blender followed by the coloring agent which is screened and added to the blender.

The powders are then compressed into tablets using appropriate conventional tools such as a suitable tableting press to form the tablet of the invention. Each tablet in the above procedure preferably contains a therapeutically effective amount of pravastatin sodium and the following excipients:

| | Weight %/Tablet |
|---|---|
| Pravastatin granules | 50-85 |
| Magnesium stearate | 0.5-2 |

Attentively, the tablets may also be formulated by a wet granulation technique where a mixture of the medicament, buffer, and osmagent is granulated using an aqueous binder solution such as polyvinyl pyrrolidone.

The tablet cores are then coated with a semipermeable membrane using a standard coating technique such as a fluidized bed coating or pan coating. The semipermeable membrane is approximately 2-30% (preferably 5-25) weight percent of the finished dosage form.

A color and/or polish coat may also be applied to the semipermeable membrane coat.

The following examples are illustrative of the present invention, and the examples should not be considered as limiting the scope of this invention in any way, as these examples and other equivalents thereof will become apparent to those versed in the art in the light of the present disclosure, and the accompanying clam.

### EXAMPLE 1

Sustained release tablet formulations containing 40 mg of pravastatin sodium are made by direct compression with the following materials in the following amounts:

| INGREDIENT | MG/TABLET | % |
|---|---|---|
| pravastatin sodium | 40 | 13.3 |
| mannitol, USP | 249.8 | 83.3 |
| sodium phosphate dibasic | 7.1 | 2.4 |
| magnesium stearate | 3 | 1 |

The pH of this tablet is approximately 7.34 and was determined by dissolving 1 tablet in 900 ml of deionized water.

These ingredients are directly compressed into tablets which then undergo pan coating with a cellulose acetate coating as follows:

| INGREDIENT | MG/TABLET | % |
|---|---|---|
| pravastatin sodium tablet, | 300 | 80 |
| 40 mg (uncoated) | | |
| cellulose acetate | 67.5 | 18 |
| polyethylene glycol | 7.5 | 2 |
| acetone | | |

The resulting cellulose acetate coated tablets are then laser drilled and pan coated with a color coating as follows:

| | MG/TABLET | % |
|---|---|---|
| pravastatin sodium tablet, | 375 | 97 |
| 40 mg (laser drilled) | | |
| Opadry White | 11.5 | 3 |
| Candelilla wax powder | 0.12 | 0.03 |

### EXAMPLE 2

Sustained release tablet formulations containing 40 mg of pravastatin sodium are made by the wet granulation process with the following ingredients in the following amounts:

| INGREDIENT | MG/TABLET | % |
|---|---|---|
| pravastatin sodium | 40 | 13.47 |
| mannitol | 249.87 | 84.13 |
| sodium phosphate dibasic | 7.13 | 2.40 |
| purified water | | |

Sodium phosphate dibasic is first dissolved in purified water. Mannitol and pravastatin are then charged into a mass mixture, and granulated with the sodium phosphate dibasic solution The granulation is then dried employing conventional methods and passed through a 30 mesh screen. The pravastatin sodium granules are then mixed with magnesium stearate and compressed into tablets as follows:

The pH of this tablet is approximately 7.35 and was determined by dissolving 1 tablet in 900 ml of deionized water.

| INGREDIENT | MG/TABLET | % |
|---|---|---|
| pravastatin sodium granules | 297 | 99 |
| magnesium stearate | 3 | 1 |

The uncoated pravastatin tablets are then coated with cellulose acetate as follows:

| INGREDIENT | MG/TABLET | % |
|---|---|---|
| pravastatin sodium tablet, | 300 | 80 |
| 40 mg (uncoated) | | |
| cellulose acetate | 67.5 | 18 |
| polyethylene glycol (PEG 400) | 75 | 2 |
| acetone | | |

The coated tablets are then laser drilled and color coated in a pan coater as follows:

| INGREDIENT | MG/TABLET | % |
|---|---|---|
| pravastatin sodium tablet, | 375 | 97 |
| 40 mg (laser drilled) | | |
| Opadry White | 11.48 | 2.97 |
| purified water | | |
| candelilla wax powder | 0.12 | 0.03 |

### EXAMPLE 3

Sustained release tablet formulations containing 40 mg of pravastatin sodium are made by wet granulation as in Example 2 with the following ingredients in the following amounts:

| INGREDIENT | MG/TABLET | % |
|---|---|---|
| pravastatin sodium | 40.00 | 13.47 |
| mannitol | 249.87 | 84.13 |
| Tromethamine | 7.13 | 0.76 |

The pH of this tablet is approximately 8.43 and was detail by dissolving 1 tablet in 900 ml of deionized water.

The pravastatin sodium granules are then mixed with magnesium stearate and compressed into tablets as follows:

| INGREDIENT | MG/TABLET | % |
|---|---|---|
| pravastatin sodium granules | 297 | 99 |
| magnesium stearate | 3 | 1 |

The uncoated pravastatin tablets are then coated with cellulose acetate as follows:

| INGREDIENT | MG/TABLET | % |
|---|---|---|
| pravastatin sodium tablet, 40 mg (uncoated) | 300.00 | 92.39 |
| cellulose acetate | 22.24 | 6.85 |
| polyethylene glycol (PEG 400) | 2.47 | 0.76 |
| acetone | | |

The coated tablets are then laser drilled and color coated in a pan coater as follows:

| INGREDIENT | MG/TABLET | % |
|---|---|---|
| pravastatin sodium tablet, | 375 | 97 |
| 40 mg (laser drilled) | | |
| Opadry White | 11.48 | 2.97 |
| purified water | | |
| candelilla wax powder | 0.12 | 0.03 |

Tablet Stability

Pravastatin sodium tablets were subjected to an accelerated stability test. The tablets were exposed to 40° C. (75% relative humidity) for three (3) months time. At the end of three (3) months time, the amount of pravastatin sodium that had degraded to lactones was determined by HPLC analysis.

The stability tests indicate that replacing the basifying agent magnesium oxide with the buffers tromethamine or sodium phosphate dibasic increases the stability of pravastatin sodium tablets with lactone not detectable and pravastatin retained at approximately 100%. There was no detectable degradation

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications and variations may be made herein, in accordance with the inventive principles disclosed, without departing from the spirit and scope of the invention.

We claim:

1. A controlled release dosage from comprising:
(a) a core comprising pravastatin or a pharmaceutically acceptable salt, isomer, or derivative thereof; and
a buffering agent
wherein the dosage form imparts a pH less than 9 when dissolved in 900 ml deionized water.

2. The dosage form of claim 1 wherein the core further comprises an osmagent.

3. The dosage form of claim 1 wherein the dosage form further comprises a coating on said core.

4. The dosage form of claim 3 wherein said coating is a semipermeable membrane.

5. The dosage form of claim 4 wherein said coating comprises a plasticizer.

6. The dosage form of claim 3 wherein said coating comprises at least one exit means.

7. The dosage form of claim 1 wherein said pravastatin is present in an amount from about 1 to 80% by weight of the composition.

8. The dosage form of claim 1 wherein said core comprises about 1-6% of said buffering agent.

9. The dosage form of claim 1 wherein the buffering agent used is tromethamine.

10. The dosage form of claim 1 wherein the buffering agent is sodium phosphate dibasic.

11. The dosage form as defined in claim 6 wherein the osmagent is selected from the group consisting of sucrose, dextrose, lactose, cellulose derivatives, povidone, calcium carbonate, calcium sulfate, magnesium carbonate, corn starch, modified corn starch, mannitol, xylitol, fructose, sorbitol and sodium chloride or mixtures thereof.

12. The dosage form of claim 1 wherein the core has a pH of less than 8.5.

13. The dosage form of claim 1 wherein the core has a pH of less than 8.0.

14. A controlled release dosage form consisting essentially of:
(a) a core with a pH of less than 9 consisting essentially of:
(i) 2-50 weight percent of pravastatin or a pharmaceutically acceptable salt, isomer, or derivitive thereof;
(ii) 30-95 weight percent of an osmagent;
(iii) 0.5-10 weight percent of a buffering agent;
(iv) 0-6 weight percent of a lubricant;
(b) a coating surrounding the core consisting essentially of:
(i) semipermeable film forming polymer and;
(ii) 0.5-20 weight percent of the coating of a plasticizer; and
(c) at least one exit means connecting the core with the exterior of the sustained release dosage form.

15. The controlled release dosage form of claim 14 wherein the core consists essentially of:
(i) 4-25 weight percent of pravastatin or a pharmaceutically acceptable salt, isomer, or derivitive thereof;
(ii) 50-80 weight percent of the osmagent;
(iii) 2-6 weight percent of a buffering agent; and
(iv) 0.5-2 weight percent of a lubricant.

16. The dosage of claim 14 wherein the core has a pH of less than 8.5.

17. The dosage of claim 14 wherein the core has a pH of less than 8.0.

18. The dosage form of claim 13 wherein said buffering agent is tromethamine.

19. The dosage form of claim 13 wherein said buffering agent is sodium diphosphate dibasic.
